(19) United States
(12) Patent Application Publication
(10) Pub. No.: US 2026/0151564 A1
(43) Pub. Date: Jun. 4, 2026

(54) Strain Relief Device For Flexible Tubing And Flow Sensor With Strain Relief For Flexible Tubing

(71) Applicant: Becton, Dickinson and Company, Franklin Lakes, NJ (US)

(72) Inventor: John Picot, Landing, NJ (US)

(21) Appl. No.: 18/965,426

(22) Filed: Dec. 2, 2024

(51) Int. Cl. A61M 5/168; A61M 5/172

(52) U.S. Cl. CPC A61M 5/16886; A61M 5/16877; A61M 5/172; A61M 2205/0216; A61M 2205/3334

(57) ABSTRACT

A flow sensor may include a flow sensor housing that extends between a first end and a second end. The second end of the flow sensor housing may include an opening to an interior of the flow sensor housing. An interior cross section of the flow sensor housing defined by an inner surface may increase from a first smaller interior cross section between the first end and the second end to a second larger interior cross section at the second end of the flow sensor housing that includes the opening. A flexible tubing may extend from the opening at the second end of the flow sensor housing. A fluid flow path of the flow sensor may be defined at least in part by the flexible tubing. The flow sensor may include at least one sensor configured to characterize at least one attribute of a fluid in the fluid flow path.

### BACKGROUND

### 1. Technical Field

This disclosure relates generally inhibiting or preventing kinking of flexible tubing and, in some non-limiting embodiments or aspects, to a flow sensor with strain relief for flexible tubing.

### 2. Technical Considerations

Flow sensors may be used to monitor and document bolus delivery to patients via intravenous (IV) tubing as part of the patients' health records. When IV tubing kinks, fluid flow within the tubing may be inhibited or completely stopped.

### SUMMARY

According to some non-limiting embodiments or aspects, provided is a flow sensor, including: a flow sensor housing that extends between a first end and a second end opposite the first end, wherein the second end of the flow sensor housing includes an opening to an interior of the flow sensor housing, and wherein an interior cross section of the interior of the flow sensor housing defined by an inner surface of the flow sensor housing increases from a first smaller interior cross section between the first end and the second end of the flow sensor housing to a second larger interior cross section at the second end of the flow sensor housing that includes the opening; a flexible tubing that extends from the opening at the second end of the flow sensor housing; a fluid flow path defined at least in part by the flexible tubing; and at least one sensor configured to characterize at least one attribute of a fluid in the fluid flow path.

In some non-limiting embodiments or aspects, the flow sensor further includes: a flow tube including a fluid inlet at a first end of the flow tube and a fluid outlet at a second end of the flow tube opposite the first end of the flow tube, wherein the fluid flow path is further defined at least in part by the flow tube; and an outlet fitting secured at a first end to the outlet of the flow tube and at a second end to the flexible tubing, wherein the flow tube is in fluid communication with the flexible tubing via the outlet fitting.

In some non-limiting embodiments or aspects, the flow sensor further includes: an inlet fitting secured at a first end to the inlet of the flow tube.

In some non-limiting embodiments or aspects, the flow sensor housing includes a plurality of ribs extending from the inner surface of the flow sensor housing into the interior of the flow sensor housing, wherein the plurality of ribs contacts and restrains a movement of the flow tube, the inlet fitting, the outlet fitting, and the flexible tubing within the flow sensor housing.

In some non-limiting embodiments or aspects, a first portion of the inner surface of the flow sensor housing that defines the interior of the flow sensor housing proximate the first smaller interior cross section is fixedly coupled to the flexible tubing.

In some non-limiting embodiments or aspects, the first portion of the inner surface of the flow sensor housing that defines the interior of the flow sensor housing proximate the first smaller interior cross section is fixedly couples to flexible tubing via at least one of the following: a press fit connection, an adhesive connection, a shrink fit connection, or any combination thereof.

In some non-limiting embodiments or aspects, the inner surface of the flow sensor housing between the first smaller interior cross section between the first end and the second end of the flow sensor housing and the second larger interior cross section at the second end of the flow sensor housing that includes the opening includes a gradually curved surface such that the interior cross section of the interior of the flow sensor housing defined by the inner surface of the flow sensor housing gradually increases from the first smaller interior cross section between the first end and the second end of the flow sensor housing to the second larger interior cross section at the second end of the flow sensor housing that includes the opening.

In some non-limiting embodiments or aspects, the gradually curved surface is configured to inhibit the flexible tubing proximate the opening at the second end of the flow sensor housing from exceeding a bending radius associated with the flexible tubing.

In some non-limiting embodiments or aspects, an exterior cross section of an exterior of the flow sensor housing defined by an exterior surface of the flow sensor housing increases from a first smaller exterior cross section between the first end and the second end of the flow sensor housing to a second larger exterior cross section at the second end of the flow sensor housing that includes the opening.

In some non-limiting embodiments or aspects, the exterior surface of the flow sensor housing between the first smaller exterior cross section between the first end and the second end of the flow sensor housing and the second larger exterior cross section at the second end of the flow sensor housing that includes the opening includes a plurality of spines extending from the exterior surface of the flow sensor housing.

According to some non-limiting embodiments or aspects, provided is a strain relief device for a flexible tubing, including: a body including a cavity that extends between a first opening at a first end of the body and a second opening at a second end of the body, wherein a first portion of an inner surface of the body that defines the cavity is configured to fixedly couple to the flexible tubing proximate the first end of the body with the flexible tubing extending through the cavity of the body and out at least the second opening at the second end of the body, and wherein a cross section of the cavity defined by the inner surface of the body increases from a first smaller cross section at the first end of the body to a second larger cross section at the second end of the body.

In some non-limiting embodiments or aspects, the inner surface of the body includes a gradually curved surface such that the cross section of the cavity defined by the inner surface of the body gradually increases from the first smaller cross section at the first end of the body to the second larger cross section at the second end of the body.

In some non-limiting embodiments or aspects, the gradually curved surface is configured to inhibit the flexible tubing proximate the second opening at the second end of the body from exceeding a bending radius associated with the flexible tubing.

In some non-limiting embodiments or aspects, the first portion of the inner surface of the body is configured to fixedly couple to the flexible tubing via at least one of the following: a press fit connection, an adhesive connection, a shrink fit connection, or any combination thereof.

According to some non-limiting embodiments or aspects, provided is a system including the strain relief device, the flexible tubing, and a medical device including a fluid flow path, wherein the flexible tubing is fixedly coupled to the first portion of the inner surface of the body that defines the cavity, and wherein the fluid flow path of the medical device is one of: directly fluidically coupled to a portion of the flexible tubing extending from the first opening at the first end of the flexible sleeve and fluidically coupled to the flexible tubing via the first opening at the first end of the body.

According to some non-limiting embodiments or aspects, provided is a strain relief device for a flexible tubing, including: a flexible sleeve including a through path that extends between a first opening at a first end of the flexible sleeve and a second opening at a second end of the flexible sleeve, wherein a first portion of an inner surface of the flexible sleeve that defines the through path is configured to fixedly couple to the flexible tubing proximate the first end of the flexible sleeve with the flexible tubing extending through the through path of the flexible sleeve and out at least the second opening at the second end of the flexible sleeve, and wherein an elastic section modulus of the flexible sleeve is greater than an elastic section modulus of the flexible tubing.

In some non-limiting embodiments or aspects, the flexible sleeve is configured to inhibit the flexible tubing extending through the through path of the flexible sleeve from exceeding a bending radius associated with the flexible tubing.

In some non-limiting embodiments or aspects, the first portion of the inner surface of the flexible tubing is configured to fixedly couple to the flexible tubing via at least one of the following: a press fit connection, an adhesive connection, a shrink fit connection, or any combination thereof.

In some non-limiting embodiments or aspects, a remaining portion of the inner surface of the flexible is configured to form a slip fit assembly with the flexible tubing extending through the through path of the flexible sleeve.

According to some non-limiting embodiments or aspects, provided is a system, including the strain relief device, the flexible tubing, and a medical device including a fluid flow path, and a pinch claim, wherein the flexible tubing is fixedly coupled to the first portion of the inner surface of the body that defines the cavity, wherein the fluid flow path of the medical device is one of: directly fluidically coupled to a portion of the flexible tubing extending from the first opening at the first end of the flexible sleeve and fluidically coupled to the flexible tubing via the first opening at the first end of the flexible sleeve, wherein the pinch clamp configured to be received over the flexible sleeve, and wherein, in response to closing the pinch clamp, the pinch clamp is configured to occlude a portion of the flexible tubing extending through the through path of the flexible sleeve.

Further non-limiting embodiments or aspects are set forth in the following numbered clauses:

Clause 1: A flow sensor, comprising: a flow sensor housing that extends between a first end and a second end opposite the first end, wherein the second end of the flow sensor housing includes an opening to an interior of the flow sensor housing, and wherein an interior cross section of the interior of the flow sensor housing defined by an inner surface of the flow sensor housing increases from a first smaller interior cross section between the first end and the second end of the flow sensor housing to a second larger interior cross section at the second end of the flow sensor housing that includes the opening; a flexible tubing that extends from the opening at the second end of the flow sensor housing; a fluid flow path defined at least in part by the flexible tubing; and at least one sensor configured to characterize at least one attribute of a fluid in the fluid flow path.

Clause 2: The flow sensor of clause 1, further comprising: a flow tube including a fluid inlet at a first end of the flow tube and a fluid outlet at a second end of the flow tube opposite the first end of the flow tube, wherein the fluid flow path is further defined at least in part by the flow tube; and an outlet fitting secured at a first end to the outlet of the flow tube and at a second end to the flexible tubing, wherein the flow tube is in fluid communication with the flexible tubing via the outlet fitting.

Clause 3: The flow sensor of clause 1 or clause 2, further comprising: an inlet fitting secured at a first end to the inlet of the flow tube.

Clause 4: The flow sensor of any of clauses 1-3, wherein the flow sensor housing includes a plurality of ribs extending from the inner surface of the flow sensor housing into the interior of the flow sensor housing, wherein the plurality of ribs contacts and restrains a movement of the flow tube, the inlet fitting, the outlet fitting, and the flexible tubing within the flow sensor housing.

Clause 5: The flow sensor of any of clauses 1-4, wherein a first portion of the inner surface of the flow sensor housing that defines the interior of the flow sensor housing proximate the first smaller interior cross section is fixedly coupled to the flexible tubing.

Clause 6: The flow sensor of any of clauses 1-5, wherein the first portion of the inner surface of the flow sensor housing that defines the interior of the flow sensor housing proximate the first smaller interior cross section is fixedly couples to flexible tubing via at least one of the following: a press fit connection, an adhesive connection, a shrink fit connection, or any combination thereof.

Clause 7: The flow sensor of any of clauses 1-6, wherein the inner surface of the flow sensor housing between the first smaller interior cross section between the first end and the second end of the flow sensor housing and the second larger interior cross section at the second end of the flow sensor housing that includes the opening includes a gradually curved surface such that the interior cross section of the interior of the flow sensor housing defined by the inner surface of the flow sensor housing gradually increases from the first smaller interior cross section between the first end and the second end of the flow sensor housing to the second larger interior cross section at the second end of the flow sensor housing that includes the opening.

Clause 8: The flow sensor of any of clauses 1-7, wherein the gradually curved surface is configured to inhibit the flexible tubing proximate the opening at the second end of the flow sensor housing from exceeding a bending radius associated with the flexible tubing.

Clause 9: The flow sensor of any of clauses 1-8, wherein an exterior cross section of an exterior of the flow sensor housing defined by an exterior surface of the flow sensor housing increases from a first smaller exterior cross section between the first end and the second end of the flow sensor housing to a second larger exterior cross section at the second end of the flow sensor housing that includes the opening.

Clause 10: The flow sensor of any of clauses 1-9, wherein the exterior surface of the flow sensor housing between the first smaller exterior cross section between the first end and the second end of the flow sensor housing and the second larger exterior cross section at the second end of the flow sensor housing that includes the opening includes a plurality of spines extending from the exterior surface of the flow sensor housing.

Clause 11: A strain relief device for a flexible tubing, comprising: a body including a cavity that extends between a first opening at a first end of the body and a second opening at a second end of the body, wherein a first portion of an inner surface of the body that defines the cavity is configured to fixedly couple to the flexible tubing proximate the first end of the body with the flexible tubing extending through the cavity of the body and out at least the second opening at the second end of the body, and wherein a cross section of the cavity defined by the inner surface of the body increases from a first smaller cross section at the first end of the body to a second larger cross section at the second end of the body.

Clause 12: The strain relief device of clause 11, wherein the inner surface of the body includes a gradually curved surface such that the cross section of the cavity defined by the inner surface of the body gradually increases from the first smaller cross section at the first end of the body to the second larger cross section at the second end of the body.

Clause 13: The strain relief device of clause 11 or clause 12, wherein the gradually curved surface is configured to inhibit the flexible tubing proximate the second opening at the second end of the body from exceeding a bending radius associated with the flexible tubing.

Clause 14: The strain relief device of any of clauses 11-13, wherein the first portion of the inner surface of the body is configured to fixedly couple to the flexible tubing via at least one of the following: a press fit connection, an adhesive connection, a shrink fit connection, or any combination thereof.

Clause 15: A system, comprising: the strain relief device of any of clauses 11-14; the flexible tubing, wherein the flexible tubing is fixedly coupled to the first portion of the inner surface of the body that defines the cavity; and a medical device including a fluid flow path, wherein the fluid flow path of the medical device is one of: directly fluidically coupled to a portion of the flexible tubing extending from the first opening at the first end of the flexible sleeve and fluidically coupled to the flexible tubing via the first opening at the first end of the body.

Clause 16: A strain relief device for a flexible tubing, comprising: a flexible sleeve including a through path that extends between a first opening at a first end of the flexible sleeve and a second opening at a second end of the flexible sleeve, wherein a first portion of an inner surface of the flexible sleeve that defines the through path is configured to fixedly couple to the flexible tubing proximate the first end of the flexible sleeve with the flexible tubing extending through the through path of the flexible sleeve and out at least the second opening at the second end of the flexible sleeve, and wherein an elastic section modulus of the flexible sleeve is greater than an elastic section modulus of the flexible tubing.

Clause 17: The strain relief device of clause 16, wherein the flexible sleeve is configured to inhibit the flexible tubing extending through the through path of the flexible sleeve from exceeding a bending radius associated with the flexible tubing.

Clause 18: The strain relief device of clause 16 or clause 17, wherein the first portion of the inner surface of the flexible tubing is configured to fixedly couple to the flexible tubing via at least one of the following: a press fit connection, an adhesive connection, a shrink fit connection, or any combination thereof.

Clause 19: The strain relief device of any of clauses 16-18, wherein a remaining portion of the inner surface of the flexible is configured to form a slip fit assembly with the flexible tubing extending through the through path of the flexible sleeve.

Clause 20: A system, comprising: the strain relief device of any of clauses 16-19; the flexible tubing, wherein the flexible tubing is fixedly coupled to the first portion of the inner surface of the body that defines the cavity; a medical device including a fluid flow path, wherein the fluid flow path of the medical device is one of: directly fluidically coupled to a portion of the flexible tubing extending from the first opening at the first end of the flexible sleeve and fluidically coupled to the flexible tubing via the first opening at the first end of the flexible sleeve; and a pinch clamp configured to be received over the flexible sleeve, wherein, in response to closing the pinch clamp, the pinch clamp is configured to occlude a portion of the flexible tubing extending through the through path of the flexible sleeve.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosed subject matter.

### BRIEF DESCRIPTION OF THE DRAWINGS

Additional advantages and details are explained in greater detail below with reference to the non-limiting, exemplary embodiments that are illustrated in the accompanying schematic figures, in which:

FIG. 1A depicts a front perspective view of a flow sensor system, according to some non-limiting embodiments or aspects;

FIG. 1B depicts a rear perspective view of a flow sensor system, according to some non-limiting embodiments or aspects;

FIG. 2A depicts a perspective view of a flow sensor of a flow sensor system, according to some non-limiting embodiments or aspects;

FIG. 2B depicts a side view of a flow sensor of a flow sensor system, according to some non-limiting embodiments or aspects;

FIG. 2C depicts a cross sectional view of a flow sensor of a flow sensor system, according to some non-limiting embodiments or aspects;

FIG. 2D depicts a cross sectional view of an outlet end of a flow sensor of a flow sensor system, according to some non-limiting embodiments or aspects;

FIG. 2E depicts a cut-away view of an outlet end of a flow sensor of a flow sensor system, according to some non-limiting embodiments or aspects;

FIGS. 2F and 2G depict side views of an outlet end of a flow sensor of a flow sensor system, according to some non-limiting embodiments or aspects;

FIGS. 2H and 21 depict cross-sectional views of an outlet end of a flow sensor of a flow sensor system, according to some non-limiting embodiments or aspects;

FIGS. 3A and 3B depict flexible tubing hanging from a flow sensor of a flow sensor system, according to some non-limiting embodiments or aspects;

FIGS. 4A and 4B depict a strain relief device for flexible tubing, according to some non-limiting embodiments or aspects;

FIGS. 5A-5C depict another strain relief device for flexible tubing, according to some non-limiting embodiments or aspects; and

FIG. 6 depicts a closed catheter system in which a strain relief device according to some non-limiting embodiments or aspects may be implemented.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the embodiments as they are oriented in the drawing figures. However, it is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects of the disclosed subject matter. Hence, specific dimensions and other physical characteristics related to the embodiments or aspects disclosed herein are not to be considered as limiting.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. In addition, reference to an action being “based on” a condition may refer to the action being “in response to” the condition. For example, the phrases “based on” and “in response to” may, in some non-limiting embodiments or aspects, refer to a condition for automatically triggering an action (e.g., a specific operation of an electronic device, such as a computing device, a processor, and/or the like).

Some of the techniques described herein may be implemented by one or more computer programs executed by one or more processors residing, for example on a medical device, such as a flow sensor, or the like. The computer programs may include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

As used herein, the term “communication” may refer to the reception, receipt, transmission, transfer, provision, and/or the like of data (e.g., information, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices. A computing device may also be a desktop computer or other form of non-mobile computer.

As used herein, the term “server” may refer to or include one or more computing devices that are operated by or facilitate communication and processing for multiple parties in a network environment, such as the Internet, although it will be appreciated that communication may be facilitated over one or more public or private network environments and that various other arrangements are possible. Further, multiple computing devices (e.g., servers, point-of-sale (POS) devices, mobile devices, etc.) directly or indirectly communicating in the network environment may constitute a “system.”

As used herein, the term “system” may refer to one or more computing devices or combinations of computing devices (e.g., processors, servers, client devices, software applications, components of such, and/or the like). Reference to “a device,” “a server,” “a processor,” and/or the like, as used herein, may refer to a previously-recited device, server, or processor that is recited as performing a previous step or function, a different device, server, or processor, and/or a combination of devices, servers, and/or processors. For example, as used in the specification and the claims, a first device, a first server, or a first processor that is recited as performing a first step or a first function may refer to the same or different device, server, or processor recited as performing a second step or a second function.

Flexible tubing, such as intravenous (IV) tubing, or the like may include an extruded elastomer tubing with a section modulus resulting from a combination of an outer diameter of the flexible tubing and a wall thickness of the flexible tubing. The material properties of the flexible tubing with the section modulus inhibit or prevent the internal diameter from collapsing when the flexible tubing is bent. For example, section modulus is a geometric property of a given cross-section used in the design of beams or flexural members, such as flexible tubing, or the like. An clastic section modulus is used to calculate a cross-section's resistance to bending within the elastic range, where stress and strain are proportional. A plastic section modulus is used to calculate a cross-section's capacity to resist bending after yielding has occurred across the entire section. The plastic section modulus is used for determining the plastic, or full moment, strength and is larger than the elastic section modulus, reflecting the section's strength beyond the elastic range.

For many applications, such as medical applications including IV therapy or the like, it is advantageous to use a flexible tubing with a section modulus resulting from a combination of an outer diameter of the tubing and a wall thickness of the tubing that provides a greatest resistance to kinking when the flexible tubing is bent under normal use while providing a satisfactory fluid flow. For example, if an IV tubing kinks, fluid flow within the IV tubing may be inhibited or shut-off altogether.

IV tubing with multiple material types and different wall thicknesses is available. For IV tubing of each material type, a thinnest wall thickness with a largest internal diameter may provide a lowest kink resistance. Although fluid flow with a largest internal diameter may be desirable, thinner wall thicknesses may fail to provide sufficient kink resistance. IV tubing may be evaluated for flexibility, tensile strength of the tubing, and bonding strength of the tubing. A preferred IV tubing may provide a greater resistance to kinking when bent while having better tensile strength and bonding strength.

The Table below lists ratings for stiffness and kink resistance of example IV tubing samples labeled A, B, C, D, and E having multiple different material types (Mat'l) with different outer diameters (OD), wall thicknesses (T), and resulting internal diameters (ID). An IV tubing may be selected for a given application according to a desired combination of properties.

TABLE
Characteristics with results:
Dimensions (in)

| Tube # | OD | ID | T | Matl | Feel | Flow | Kink | Restriction |
|---|---|---|---|---|---|---|---|---|
| A | 0.145 | 0.079 | 0.033 | 80A | soft | Best | some | reduced |
| B | 0.145 | 0.039 | 0.053 | 80A | soft | Good | slight | unchanged |
| C | 0.145 | 0.039 | 0.053 | 92A | firm | Good | slight | unchanged |
| D | 0.145 | 0.079 | 0.033 | 92A | firm | Best | some | reduced |
| E | 0.145 | 0.063 | 0.041 | 92A | firm | Better | less | reduced |

A bend or bending radius, which is measured to the inside curvature, is the minimum radius one can bend a pipe, tube, sheet, cable, or hose without kinking it, damaging it, or shortening its life. The smaller the bend radius, the greater the material flexibility (as the radius of curvature decreases, the curvature increases). For example, a bending radius of a flexible tubing may be a result of bending the flexible tubing while restraining an end of the flexible tubing to a smallest internal radius at which an internal opening of the flexible tubing is not restricted. Eventually all hollow tubing will collapse when the minimum bend radius falls below the safe dimension.

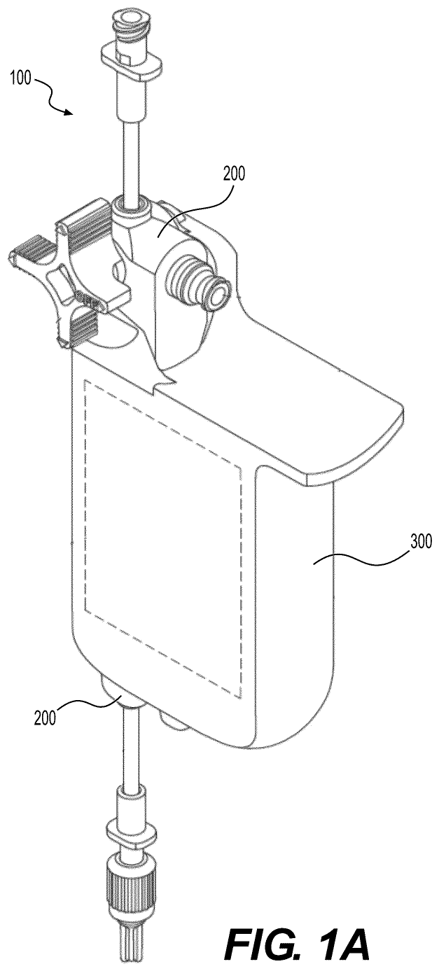

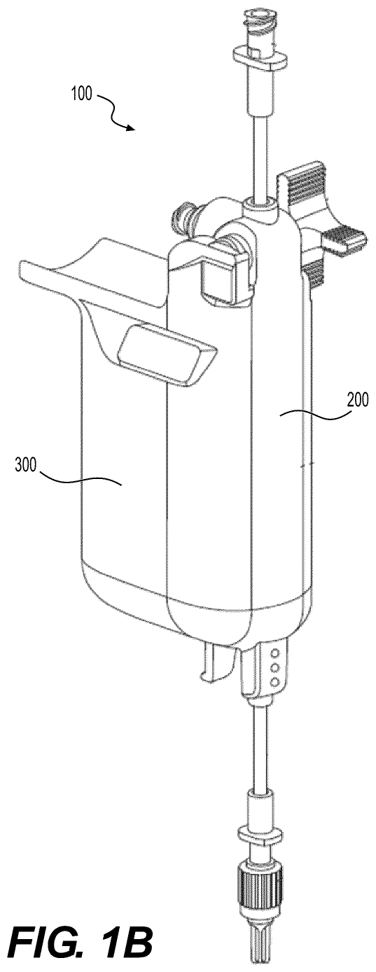

Referring now to FIGS. 1A and 1B, flow sensor system 100 may include two main assemblies which fit together prior to use: flow sensor 200 and base 300. In some non-limiting embodiments or aspects, flow sensor 200 may be a single-use flow sensor which is engageable with reusable base 300. In some non-limiting embodiments or aspects, flow sensor system 100 includes a flow sensor system as described in U.S. Provisional Patent Application No. 63/589,132, filed Oct. 10, 2023, or U.S. Provisional Patent Application No. 63/589,115, filed on Oct. 10, 2023, which are incorporated herein by reference in their entirety.

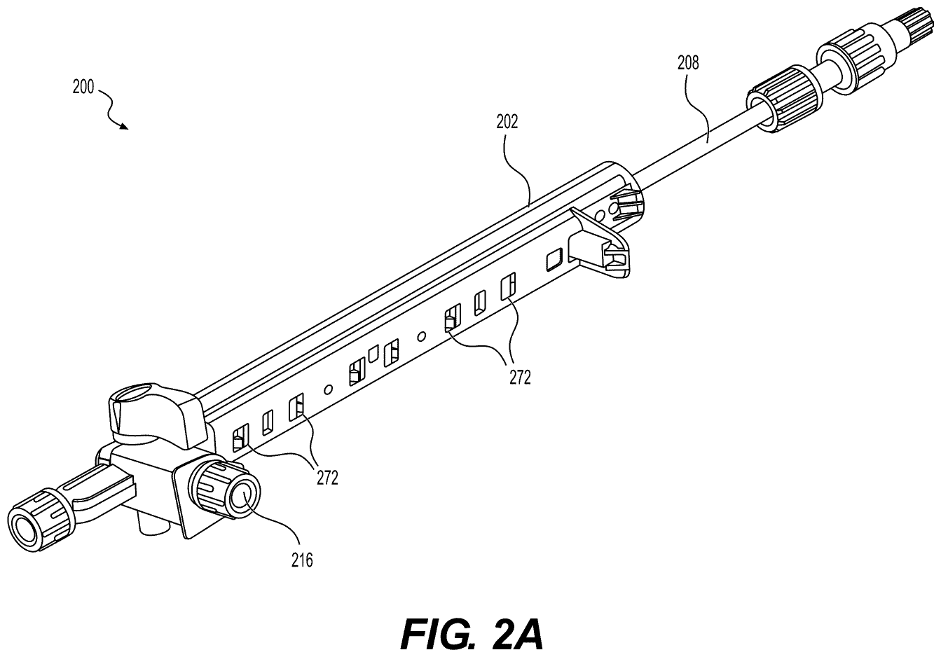

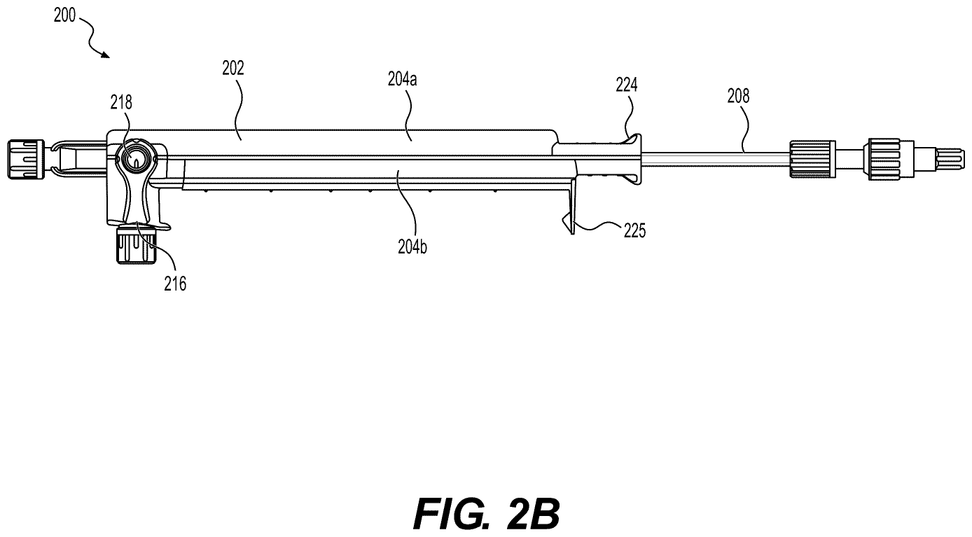

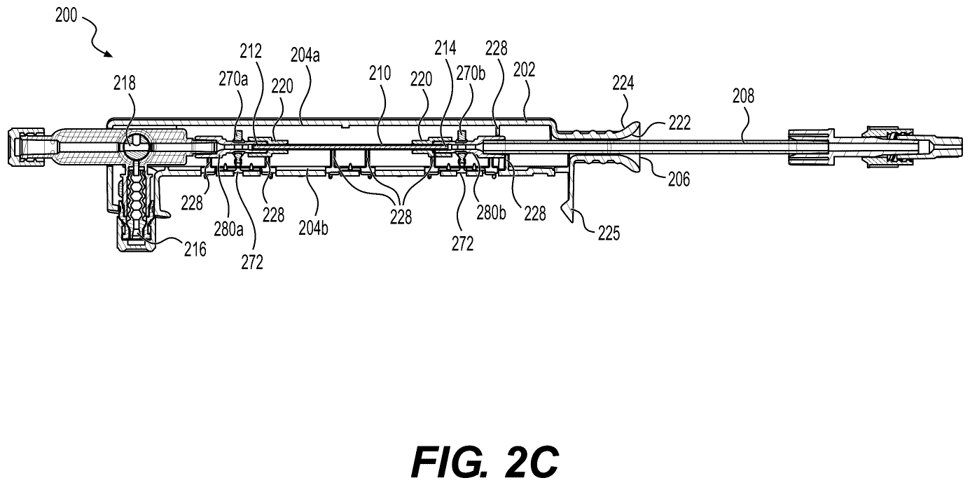

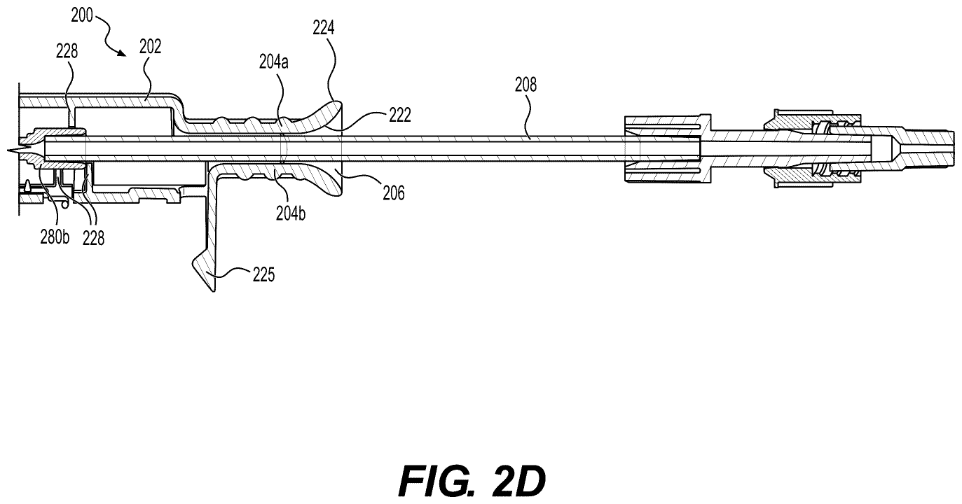

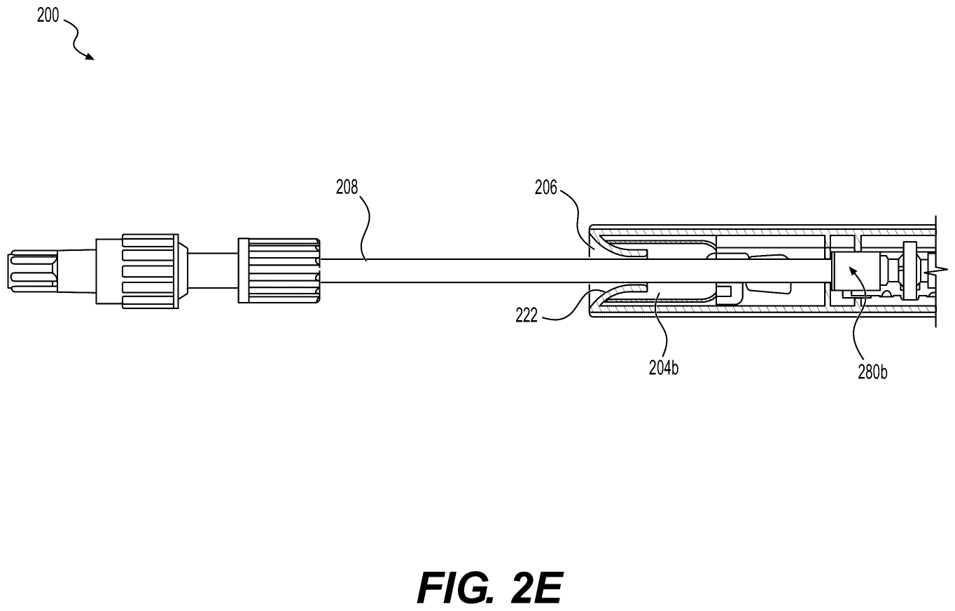

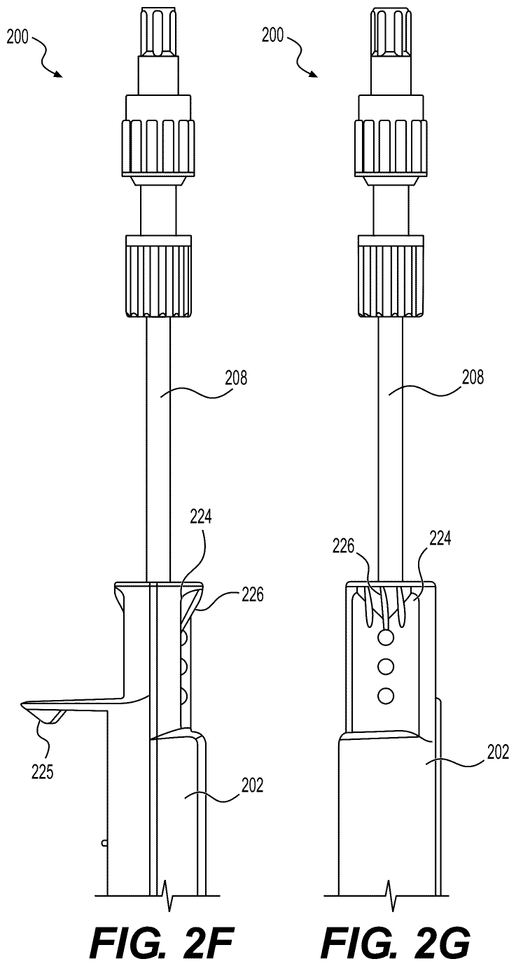

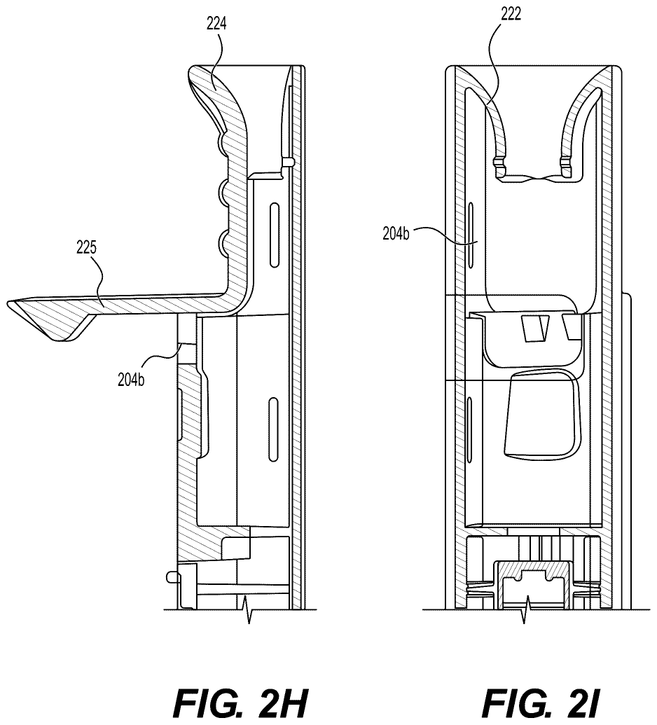

Referring now to FIGS. 2A-21, flow sensor 200 may include flow sensor housing 202 that extends between a first end and a second end opposite the first end. In some implementations, flow sensor housing 202 may include first housing portion 204a and second housing portion 204b that are connected together with components of flow sensor 200 housed therewithin. The second end of flow sensor housing 202 may include opening 206 to an interior of flow sensor housing 202. Flexible tubing 208 may extend from opening 206 at the second end of flow sensor housing 202. Flow sensor 200 may include a fluid flow path that may be defined at least in part by flexible tubing 208. The fluid flow path of flow sensor 200 may be further defined at least in part by flow tube 210. Flow tube 210 may include fluid inlet 212 at a first end of flow tube 210 and fluid outlet 214 at a second end of flow tube 210 opposite the first end of flow tube 210. Flow sensor 200 may include fluid injection port 216 and valve 218 (e.g., a manual stopcock valve, etc.) configured to control a flow of a fluid in the fluid flow path of flow sensor 200.

Flow sensor 200 may include at least one sensor 270 configured to characterize at least one attribute of a fluid in the fluid flow path of flow sensor 200 (e.g., in flow tube 210, etc.). Flow sensor electrical contact 272 (e.g., a plurality of flow sensor electrical contacts 272, etc.) may be in electrical communication with the at least one sensor 270. The at least one sensor 270 may include first ultrasonic transducer or piezoelectric element 270 arranged at an upstream position of the fluid flow path of flow sensor 200 and second ultrasonic transducer or piezoelectric element 270b arranged at a downstream position of the fluid flow path of flow sensor 200. First and second ultrasonic transducers or piezoelectric elements 270a, 270b may be configured to transmit a flow signal indicative of a flow of a fluid (e.g., a flow rate of a fluidic medicament, etc.) in the fluid flow path of flow sensor 200 (e.g., in flow tube 210, etc.). First ultrasonic transducer or piezoelectric element 270a and second ultrasonic transducer or piezoelectric element 270b may be annular in shape and encircle the fluid flow path of flow sensor 200 at respective mounting points a pre-selected distance from each other.

Flow sensor 200 may include inlet fitting or coupler 280a secured to fluid inlet 212 of flow tube 210 and outlet fitting or coupler 280b secured to fluid outlet 214 of flow tube 210. For example, inlet fitting or coupler 280a may be secured at a first end to fluid inlet 212 of flow tube 210 and at a second end opposite the first end to an outlet of valve 218, and/or outlet fitting or coupler 280b may be secured at a first end to fluid outlet 214 of flow tube 210 and at a second end to flexible tubing 208. For example, flow tube 210 may be in fluid communication with valve 218 via inlet fitting 280a, and/or flow tube 210 may be in fluid communication with flexible tubing 208 via outlet fitting or coupler 280b. As an example, flexible tubing 208 may be fixedly coupled to the second end of outlet fitting or coupler 280b.

First ultrasonic transducer or piezoelectric element 270a may be secured to or mounted on inlet fitting or coupler 280a and/or second ultrasonic transducer or piezoelectric element 270b may be secured to or mounted on outlet fitting or coupler 280b. For example, first ultrasonic transducer or piezoelectric element 270a may be annular in shape and encircle inlet fitting or coupler 280a and/or second ultrasonic transducer or piezoelectric element 270b may be annular in shape and encircle outlet fitting or coupler 280b. At least one absorber sleeve 220 may be engaged with (e.g., directly engaged with, etc.) and/or encircle inlet fitting or coupler 280a, flow tube 210, and/or outlet fitting or coupler 280b.

First and second ultrasonic transducers or piezoelectric elements 270a and 270b may be in electrical communication with one or more processors of base 300 when flow sensor 200 is connected to base 300. For example, base 300 may interact with first and second ultrasonic transducers or piezoelectric elements 270a and 270b in flow sensor 200 to measure displacement of fluid through flow sensor 200. For example, flow sensor electrical contact 272 may be in electrical communication with a corresponding base electrical contact when flow sensor 200 is connected (e.g., connected, attached, mounted, etc.) to base 300. Flow sensor 200 and base 300 may be detachably connectable to each other via a snap-fit connection between flow sensor housing 202 and base 300. For example, flow sensor housing 202 may include beam 225 that extends from flow sensor housing 202 between the first end and the second end of flow sensor housing 202 (e.g., that extends from flow sensor housing 202 distal or downstream of outlet fitting or coupler 280b within flow sensor housing 202, etc.). Beam 225 may be configured to form the snap-fit connection with base 300 as described in U.S. Provisional Patent Application No. 63/589,132, filed Oct. 10, 2023, or U.S. Provisional Patent Application No. 63/589,115, filed on Oct. 10, 2023, which are incorporated herein by reference in their entirety.

At the second end of flow sensor housing 202 that includes opening 206 to the interior of flow sensor housing 202, an interior cross section of the interior of flow sensor housing 202 may be defined by inner surface 222 of flow sensor housing 202 that increases from a first smaller interior cross section between the first end and the second end of flow sensor housing 202 (e.g., from a first smaller cross section distal or downstream of outlet fitting or coupler 280b at which inner surface 222 is in direct and/or fixed contact with flexible tubing 208, from a first smaller cross section proximate, distal or downstream of beam 225 at which inner surface 222 is in direct and/or fixed contact with flexible tubing 208, etc.) to a second larger interior cross section at the second end of flow sensor housing 202 that includes opening 206. For example, inner surface 222 of flow sensor housing 202 between the first smaller interior cross section between the first end and the second end of flow sensor housing 202 and the second larger interior cross section at the second end of flow sensor housing 202 that includes opening 206 may include a gradually curved surface such that the interior cross section of the interior of the flow sensor housing 202 defined by inner surface 222 of flow sensor housing 202 gradually increases from the first smaller interior cross section between the first end and the second end of flow sensor housing 202 to the second larger interior cross section at the second end of flow sensor housing 202 that includes opening 206. For example, inner surface 222 may gradually extend away from a central axis flow tube 210 and/or flow sensor housing 202. As an example, the interior cross section of the interior of flow sensor housing 202 defined by inner surface 222 may have a conical shape with a wider opening at opening 206 at the second end of flow sensor housing 202 and a narrower opening at the first smaller interior cross section between the first end and the second end of flow sensor housing 202. The gradually curved surface of inner surface 222 may be configured to inhibit or prevent flexible tubing 208 proximate opening 206 at the second end of flow sensor housing 202 from exceeding a bending radius associated with flexible tubing 208 (e.g., inhibit or prevent flexible tubing 208 from kinking proximate opening 206 at the second end of flow sensor housing 202, etc.). Accordingly, flow sensor housing 202 may provide 360-degree strain relief for flexible tubing 208 that inhibits or prevents bending of flexible tubing 208 that exceeds exceeding a minimum bending radius of flexible tubing 208, thereby inhibiting or preventing kinking of flexible tubing 208 proximate opening 206.

An exterior cross section of an exterior of flow sensor housing 202 defined by exterior surface 224 of flow sensor 202 housing may increase from a first smaller exterior cross section between the first end and the second end of flow sensor housing 202 to a second larger exterior cross section at the second end of flow sensor housing 202 that includes the opening 206. Exterior surface 224 of flow sensor housing 202 between the first smaller exterior cross section between the first end and the second end of flow sensor housing 202 and the second larger exterior cross section at the second end of the flow sensor housing 202 that includes opening 206 may include a plurality of spines 226 extending from exterior surface 224 of flow sensor housing 202. For example, exterior surface 224 of flow sensor housing 202 between the first smaller exterior cross section between the first end and the second end of flow sensor housing 202 and the second larger exterior cross section at the second end of the flow sensor housing 202 that includes opening 206 may be configured as a protruding grip at the second end of flow sensor housing 202. For example, disconnection of flow sensor 200 from base 300 may be achieved via the protruding grip by grasping the protruding grip and swinging the protruding grip away from base 300. The plurality of spines 226 may further define a grip profile for thumb and index fingers while maintaining wall thickness for the kink-resistant features provided by flow sensor housing 202.

The first portion of inner surface 222 of flow sensor housing 202 that defines the interior of flow sensor housing 202 proximate the first smaller interior cross section (e.g., from the first smaller cross section distal or downstream of outlet fitting or coupler 280b at which inner surface 222 is in direct and/or fixed contact with flexible tubing 208, from the first smaller cross section proximate, distal or downstream of beam 225 at which inner surface 222 is in direct and/or fixed contact with flexible tubing 208, etc.) may be fixedly coupled to flexible tubing 208. For example, the first portion of inner surface 222 of flow sensor housing 202 that defines the interior of flow sensor housing proximate 202 the first smaller interior cross section may be fixedly coupled to flexible tubing 208 via at least one of the following: a press fit connection, an adhesive connection, a shrink fit connection, or any combination thereof. Flexible tubing 208 may be free to move within or relative to the larger interior cross section (e.g., within the gradually increasing interior cross section, etc.) distal or downstream of the first smaller cross section at which inner surface 222 is fixedly coupled to flexible tubing 208.

Flow sensor housing 202 may include a plurality of ribs 228 extending from inner surface 222 of flow sensor housing 202 into the interior of flow sensor housing 202. The plurality of ribs 228 may contact and restrain a movement of flow tube 210, inlet fitting or coupler 280a, outlet fitting or coupler 280b, and/or flexible tubing 208 within flow sensor housing 202. For example, the plurality of ribs 228 may extend from first housing portion 204a and second housing portion 204b with clearance cutouts corresponding to components of flow sensor 200 within flow sensor housing 202 such that when first housing portion 204a and second housing portion 204b are connected together with the components of flow sensor 200 housed therewithin the components of flow sensor 200 housed therewithin are fixed relative to and supported by flow sensor housing 202 by the plurality of ribs 228.

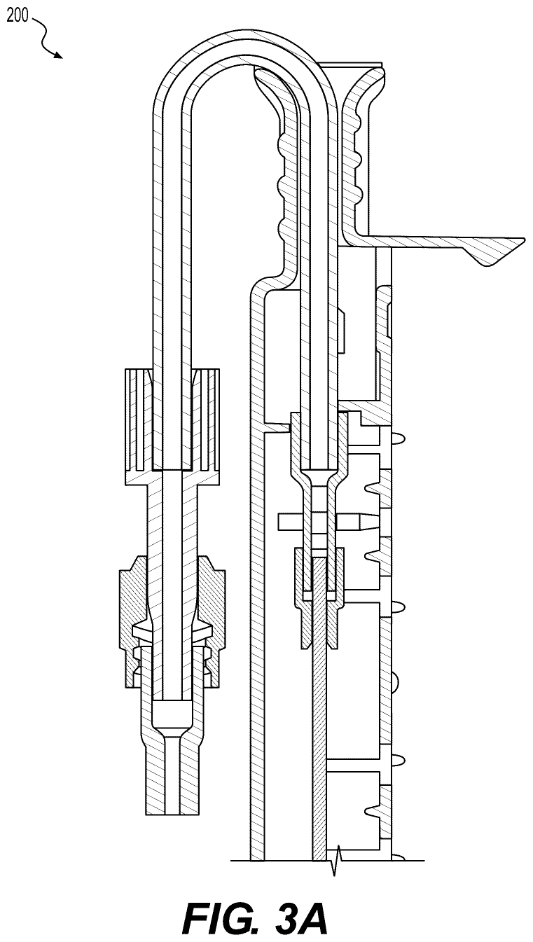

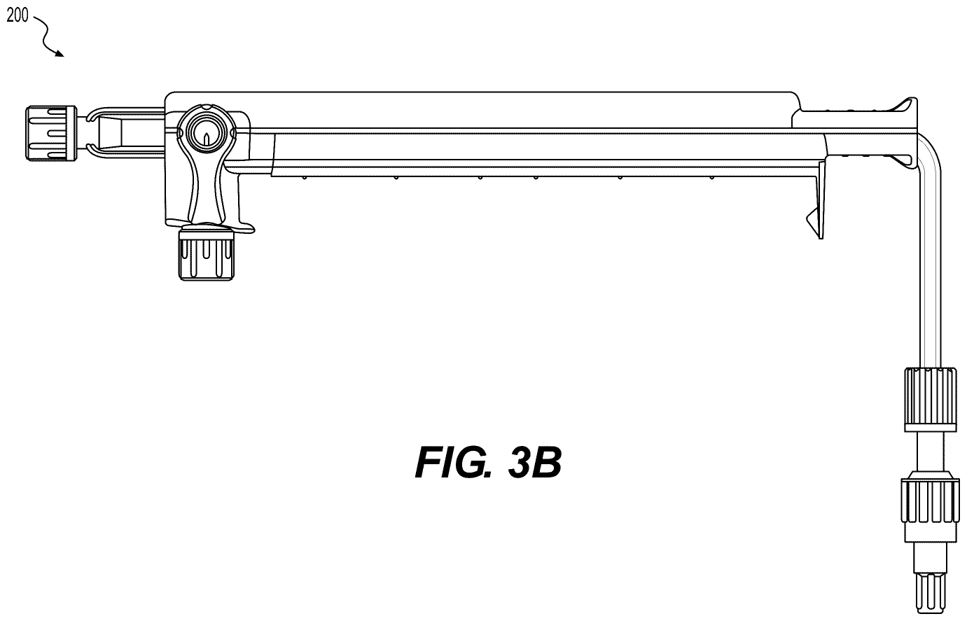

Flow sensor system 100 (e.g., base 300 with flow sensor 200 connected thereto, etc.) may be positioned vertically or horizontally on an IV pole (e.g., using a mounting cradle and clamp assembly, etc.) or positioned on a table near a patient. In such scenarios, and referring specifically to FIGS. 3A and 3B, flexible tubing 208 may drop under its own weight and hang from flow sensor 200. When flexible tubing 208 is bent, overflow sensor housing 202 proximate opening 206, the cross section of the sensor housing strain relief may inhibit bending of flexible tubing from exceeding the bending radius of flexible tubing 208, thereby inhibiting or preventing kinking of flexible tubing 208. Accordingly, flexible tubing 208 may not kink when free hanging IV tubing is suspended from flow sensor housing 202.

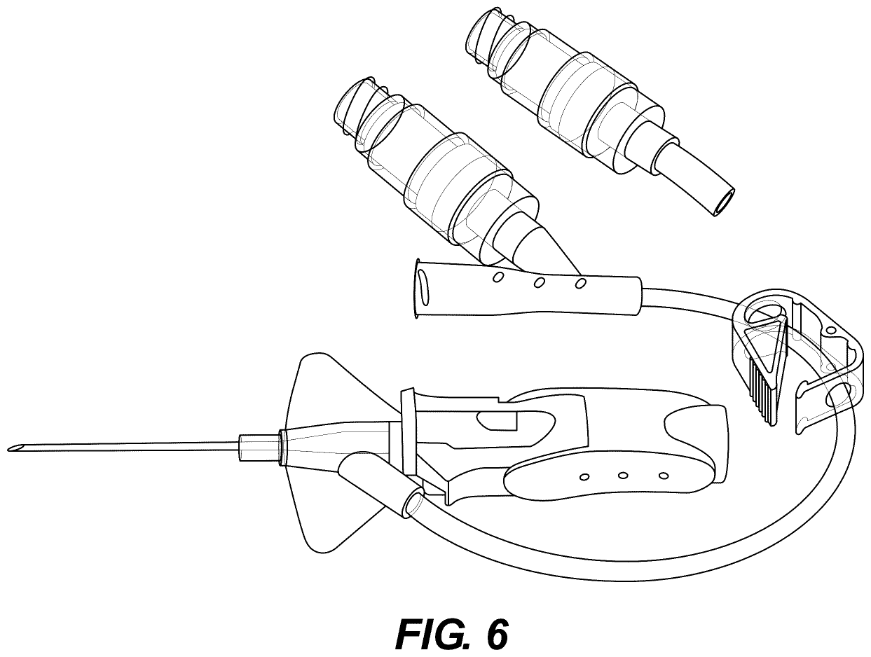

Although described herein primarily with respect to flow sensor system 100, a combination of flexible tubing with a desired or an optimized geometry cross section and material properties for enhanced kink resistance combined with or without mechanical strain relief can be used with any system incorporating flexible tubing in which kinking may cause restricted fluid flow. For example, and referring also to FIG. 6, kink resistance may be advantageous at any IV connection (e.g., in a closed catheter system, etc.) where a rigid part of an assembly is restricted from movement at a location at which the IV tubing is redirected externally, thereby causing interface stress and a bend in the IV tubing at the interface of the rigid component. Providing a more kink resistant IV tubing may reduce incidents of kinking of fluid restriction, and including a strain relief device may provide support for the IV tubing by providing a bend radius to support the IV tubing at a location at which a sharp transition may cause the IV tubing to kink at a sharp edge at the projection.

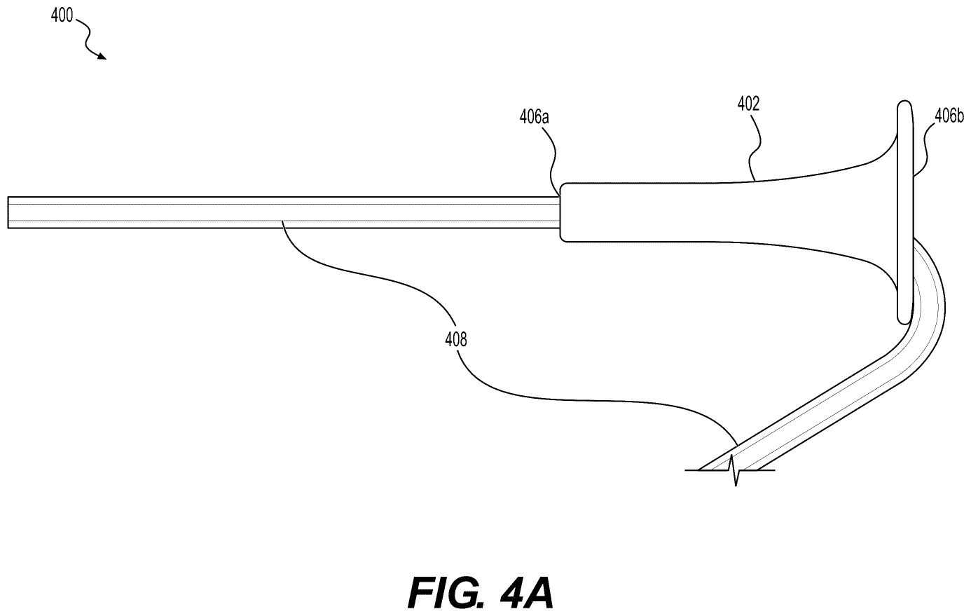

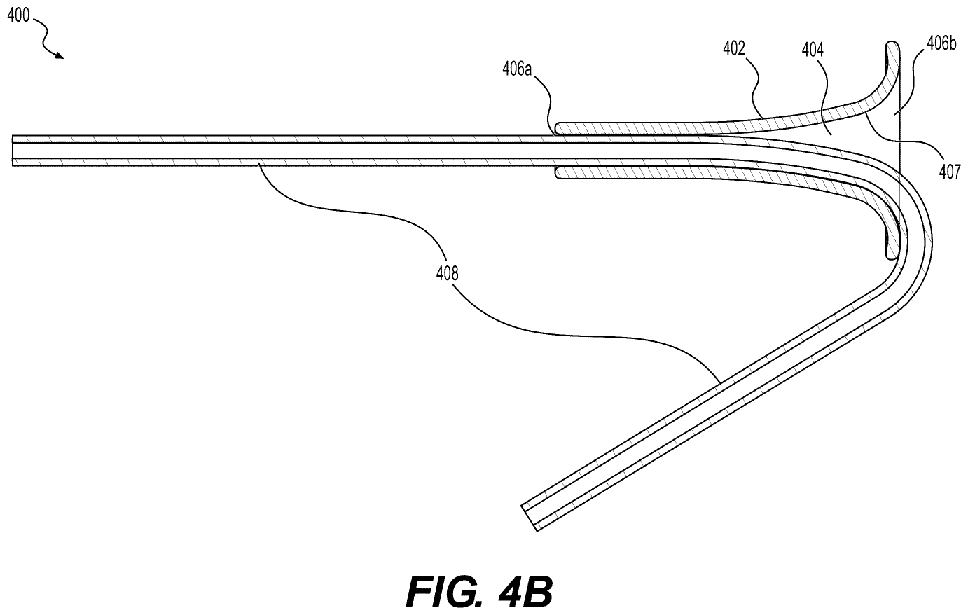

Referring now to FIGS. 4A and 4B, strain relief device 400 may include body 402 including cavity 404 that extends between first opening 406a at a first end of body 402 and second opening 406b at a second end of body 402. A first portion of inner surface 407 of body 402 that defines cavity 404 may be configured to fixedly couple to flexible tubing 408 proximate the first end of body 402 with flexible tubing 408 extending through cavity 404 of body 402 and out at least second opening 406b at the second end of body 402. For example, the first portion of the inner surface 407 of body 402 may be configured to fixedly couple to flexible tubing 408 via at least one of the following: a press fit connection, an adhesive connection, a shrink fit connection, or any combination thereof. A remaining portion of inner surface 407 of body 402 that defines cavity 404 may not be configured to fixedly couple to flexible tubing 408 downstream of the first portion and/or proximate the second end of body 402 with flexible tubing 408 extending through cavity 404 of body 402 and out at least second opening 406b at the second end of body 402. For example, flexible tubing 408 may be not fixedly restrained with respect to body 402 proximate second opening 406b at the second end of body 402.

A cross section of cavity 404 defined by inner surface 407 of body 402 may increase from a first smaller cross section at the first end of body 402 to a second larger cross section at the second end of body 402. For example, inner surface 407 of body 402 may include a gradually curved surface (e.g., a funnel shaped surface, etc.) such that the cross section of cavity 404 (e.g., an inner diameter of cavity 404, etc.) defined by inner surface 407 of body 402 gradually increases from the first smaller cross section (e.g., a first smaller diameter, etc.) at the first end of body 402 to the second larger cross section (e.g., a second larger diameter, etc.) at the second end of body 402. As an example, the gradually curved surface of inner surface 408 may be configured to inhibit flexible tubing 408 proximate second opening 406b at the second end of body 402 from exceeding a bending radius associated with flexible tubing 408.

Some non-limiting embodiments or aspects of strain relief device 400 may be incorporated into or used with a medical device (e.g., a needleless connector, a catheter, an IV sharp, etc.) including a fluid flow path (e.g., a fluid flow path at least partially defined by or fluidically connected to flexible tubing 408, etc.), and the fluid flow path of the medical device may be one of: directly fluidically coupled to a portion of flexible tubing 408 extending from first opening 406a at the first end of body 402 and fluidically coupled to flexible tubing 402 via the first opening 406a at the first end of body 402.

Accordingly, some non-limiting embodiments or aspects of the present disclosure may provide a strain relief device with a curved surface configured to control a bend radius to support an IV tubing of a medical device up to a desired bending angle orientation, which may include a right angle (90-degree) bend or a complete reversal (180 degree) bend of the IV tubing.

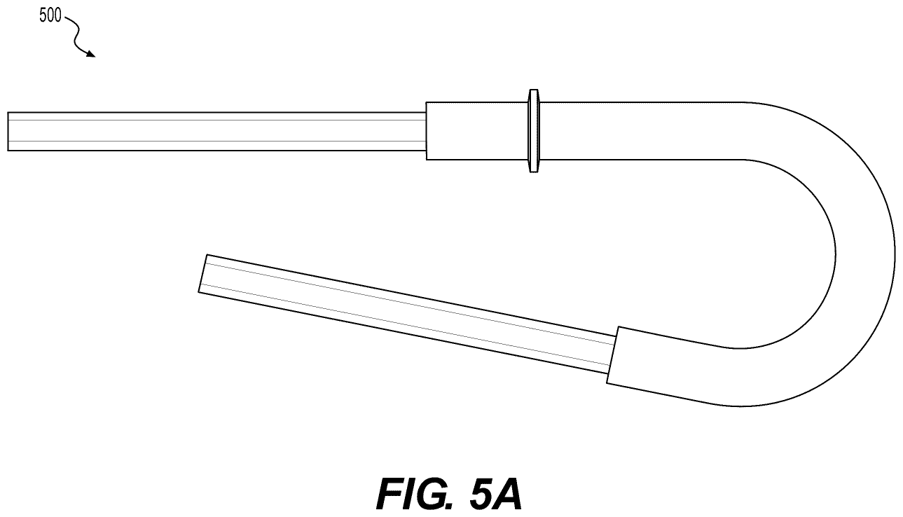

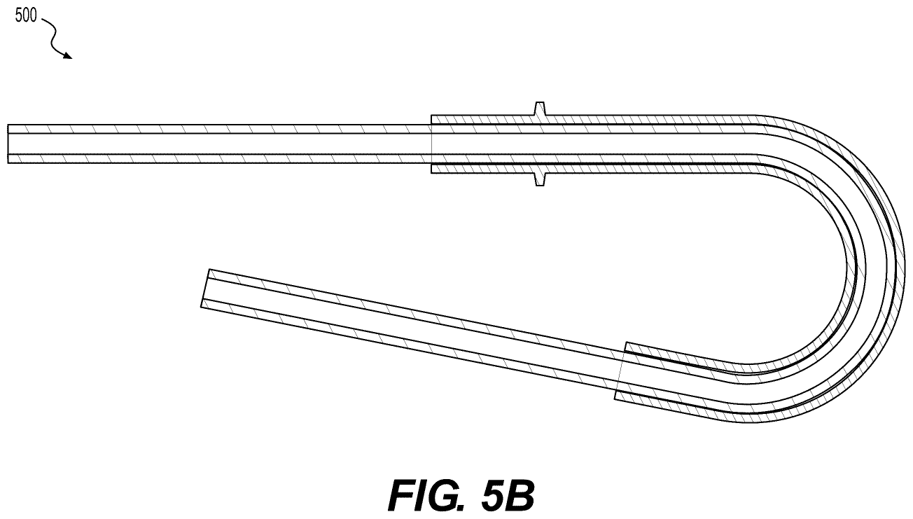

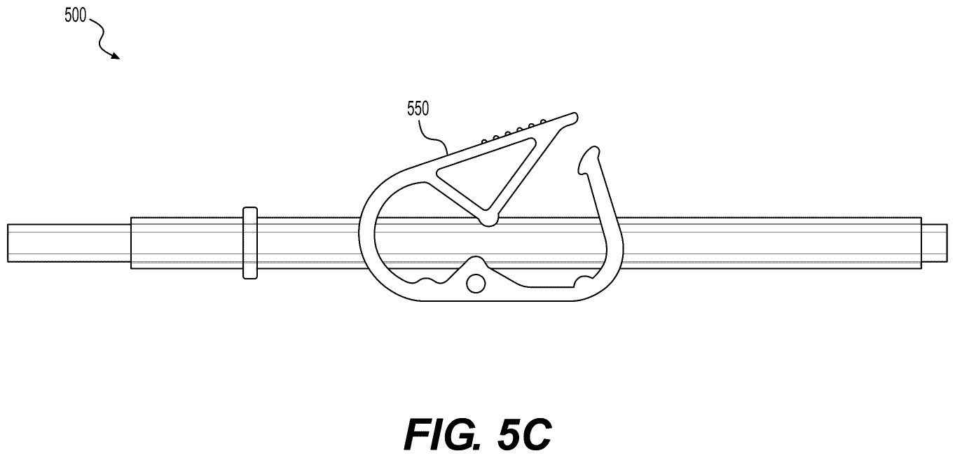

Referring now to FIGS. 5A-5C, strain relief device 500 may include flexible sleeve 502 including through path 504 that extends between first opening 506a at a first end of flexible sleeve 502 and second opening 506b at a second end of flexible sleeve 502. A first portion of inner surface 507 of flexible sleeve 502 that defines through path 504 may be configured to fixedly couple to flexible tubing 508 proximate the first end of flexible sleeve 502 with flexible tubing 508 extending through the through path of the flexible sleeve and out at least second opening 506b at the second end of flexible sleeve 502. For example, the first portion of inner surface 507 of flexible sleeve 502 may be configured to fixedly couple to flexible tubing 508 via at least one of the following: a press fit connection, an adhesive connection, a shrink fit connection, or any combination thereof. A remaining portion of inner surface 507 of flexible sleeve 502 may be configured to form a slip fit assembly with flexible tubing 508 extending through path 504 of flexible sleeve 502. For example, the remaining portion of inner surface 507 of flexible sleeve 502 that defines through path 504 may not be configured to fixedly couple to flexible tubing 508 downstream of the first portion and/or proximate the second end of flexible sleeve 502 with flexible tubing 508 extending through path 502 of flexible sleeve 502 and out at least second opening 506b at the second end of flexible sleeve 502.

An elastic section modulus of flexible sleeve 502 may be greater than an elastic section modulus of flexible tubing 508. For example, flexible sleeve 502 may be configured to inhibit flexible tubing 508 extending through path 504 of flexible sleeve 502 from exceeding a bending radius associated with flexible tubing 502.

Some non-limiting embodiments or aspects of strain relief device 500 may be incorporated into or used with a medical device (e.g., a needleless connector, a catheter, an IV sharp, etc.) including a fluid flow path (e.g., a fluid flow path at least partially defined by or fluidically connected to flexible tubing 508, etc.), and the fluid flow path of the medical device may be one of: directly fluidically coupled to a portion of flexible tubing 408 extending from first opening 506a at the first end of flexible sleeve 502 and fluidically coupled to flexible tubing 502 via the first opening 506a at the first end of flexible sleeve 502.

Accordingly, some non-limiting embodiments or aspects of the present disclosure may provide a strain relief device with a flexible sheath that is stronger or stiffer than an IV tubing itself and with predefined stiffening strength limit that enables controlled bending of the flexible sheath and the IV tubing when the IV tubing is pulled at an angle, thereby providing a controlled bending radius.

Referring now specifically to FIG. 5C, non-limiting embodiments or aspects of strain relief device 500 may be used with pinch clamp 550. Pinch clamp 550 may be configured to be received over flexible sleeve 502. In response to closing pinch clamp 550, pinch clamp 550 may be configured to occlude a portion of flexible tubing 508 extending through path 504 of flexible sleeve 502. Pinch clamp 550 may be adhered at a location along flexible sleeve 502 or slidable along a length of flexible sleeve 502 and/or flexible tubing 508. In some non-limiting embodiments or aspects, pinch clamp 550 may include a pinch clamp device as described by U.S. Pat. No. 11,040,186, filed on Oct. 5, 2016, which is incorporated herein by reference in its entirety.

Accordingly, some non-limiting embodiments or aspects of the present disclosure may provide for combining sleeve strain relief with IV tubing and clamping to provide further kink resistance in bending at a location at which the clamp is positioned on the sleeve. For example, the pinch clamp may function to restrict fluid flow in the IV tubing when closed because the IV tubing and flexible sleeve may be compressed with normal clamping force.

Although embodiments have been described in detail for the purpose of illustration, it is to be understood that such detail is solely for that purpose and that the disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

What is claimed is:

1. A flow sensor, comprising:
a flow sensor housing that extends between a first end and a second end opposite the first end, wherein the second end of the flow sensor housing includes an opening to an interior of the flow sensor housing, and wherein an interior cross section of the interior of the flow sensor housing defined by an inner surface of the flow sensor housing increases from a first smaller interior cross section between the first end and the second end of the flow sensor housing to a second larger interior cross section at the second end of the flow sensor housing that includes the opening;
a flexible tubing that extends from the opening at the second end of the flow sensor housing;
a fluid flow path defined at least in part by the flexible tubing; and
at least one sensor configured to characterize at least one attribute of a fluid in the fluid flow path.

2. The flow sensor of claim 1, further comprising:
a flow tube including a fluid inlet at a first end of the flow tube and a fluid outlet at a second end of the flow tube opposite the first end of the flow tube, wherein the fluid flow path is further defined at least in part by the flow tube; and
an outlet fitting secured at a first end to the outlet of the flow tube and at a second end to the flexible tubing, wherein the flow tube is in fluid communication with the flexible tubing via the outlet fitting.

3. The flow sensor of claim 2, further comprising:
an inlet fitting secured at a first end to the inlet of the flow tube.

4. The flow sensor of claim 1, wherein the flow sensor housing includes a plurality of ribs extending from the inner surface of the flow sensor housing into the interior of the flow sensor housing, wherein the plurality of ribs contacts and restrains a movement of the flow tube, the inlet fitting, the outlet fitting, and the flexible tubing within the flow sensor housing.

5. The flow sensor of claim 1, wherein a first portion of the inner surface of the flow sensor housing that defines the interior of the flow sensor housing proximate the first smaller interior cross section is fixedly coupled to the flexible tubing.

6. The flow sensor of claim 5, wherein the first portion of the inner surface of the flow sensor housing that defines the interior of the flow sensor housing proximate the first smaller interior cross section is fixedly couples to flexible tubing via at least one of the following: a press fit connection, an adhesive connection, a shrink fit connection, or any combination thereof.

7. The flow sensor of claim 1, wherein the inner surface of the flow sensor housing between the first smaller interior cross section between the first end and the second end of the flow sensor housing and the second larger interior cross section at the second end of the flow sensor housing that includes the opening includes a gradually curved surface such that the interior cross section of the interior of the flow sensor housing defined by the inner surface of the flow sensor housing gradually increases from the first smaller interior cross section between the first end and the second end of the flow sensor housing to the second larger interior cross section at the second end of the flow sensor housing that includes the opening.

8. The flow sensor of claim 7, wherein the gradually curved surface is configured to inhibit the flexible tubing proximate the opening at the second end of the flow sensor housing from exceeding a bending radius associated with the flexible tubing.

9. The flow sensor of claim 1, wherein an exterior cross section of an exterior of the flow sensor housing defined by an exterior surface of the flow sensor housing increases from a first smaller exterior cross section between the first end and the second end of the flow sensor housing to a second larger exterior cross section at the second end of the flow sensor housing that includes the opening.

10. The flow sensor of claim 9, wherein the exterior surface of the flow sensor housing between the first smaller exterior cross section between the first end and the second end of the flow sensor housing and the second larger exterior cross section at the second end of the flow sensor housing that includes the opening includes a plurality of spines extending from the exterior surface of the flow sensor housing.

11. A strain relief device for a flexible tubing, comprising:
a body including a cavity that extends between a first opening at a first end of the body and a second opening at a second end of the body, wherein a first portion of an inner surface of the body that defines the cavity is configured to fixedly couple to the flexible tubing proximate the first end of the body with the flexible tubing extending through the cavity of the body and out at least the second opening at the second end of the body, and wherein a cross section of the cavity defined by the inner surface of the body increases from a first smaller cross section at the first end of the body to a second larger cross section at the second end of the body.

12. The strain relief device of claim 11, wherein the inner surface of the body includes a gradually curved surface such that the cross section of the cavity defined by the inner surface of the body gradually increases from the first smaller cross section at the first end of the body to the second larger cross section at the second end of the body.

13. The strain relief device of claim 12, wherein the gradually curved surface is configured to inhibit the flexible tubing proximate the second opening at the second end of the body from exceeding a bending radius associated with the flexible tubing.

14. The strain relief device of claim 11, wherein the first portion of the inner surface of the body is configured to fixedly couple to the flexible tubing via at least one of the following: a press fit connection, an adhesive connection, a shrink fit connection, or any combination thereof.

15. A system, comprising:
the strain relief device of claim 11;
the flexible tubing, wherein the flexible tubing is fixedly coupled to the first portion of the inner surface of the body that defines the cavity; and
a medical device including a fluid flow path, wherein the fluid flow path of the medical device is one of: directly fluidically coupled to a portion of the flexible tubing extending from the first opening at the first end of the flexible sleeve and fluidically coupled to the flexible tubing via the first opening at the first end of the body.

16. A strain relief device for a flexible tubing, comprising:
a flexible sleeve including a through path that extends between a first opening at a first end of the flexible sleeve and a second opening at a second end of the flexible sleeve, wherein a first portion of an inner surface of the flexible sleeve that defines the through path is configured to fixedly couple to the flexible tubing proximate the first end of the flexible sleeve with the flexible tubing extending through the through path of the flexible sleeve and out at least the second opening at the second end of the flexible sleeve, and wherein an elastic section modulus of the flexible sleeve is greater than an elastic section modulus of the flexible tubing.

17. The strain relief device of claim 16, wherein the flexible sleeve is configured to inhibit the flexible tubing extending through the through path of the flexible sleeve from exceeding a bending radius associated with the flexible tubing.

18. The strain relief device of claim 16, wherein the first portion of the inner surface of the flexible tubing is configured to fixedly couple to the flexible tubing via at least one of the following: a press fit connection, an adhesive connection, a shrink fit connection, or any combination thereof.

19. The strain relief device of claim 16, wherein a remaining portion of the inner surface of the flexible is configured to form a slip fit assembly with the flexible tubing extending through the through path of the flexible sleeve.

20. A system, comprising:
the strain relief device of claim 16;
the flexible tubing, wherein the flexible tubing is fixedly coupled to the first portion of the inner surface of the body that defines the cavity;
a medical device including a fluid flow path, wherein the fluid flow path of the medical device is one of: directly fluidically coupled to a portion of the flexible tubing extending from the first opening at the first end of the flexible sleeve and fluidically coupled to the flexible tubing via the first opening at the first end of the flexible sleeve; and
a pinch clamp configured to be received over the flexible sleeve, wherein, in response to closing the pinch clamp, the pinch clamp is configured to occlude a portion of the flexible tubing extending through the through path of the flexible sleeve.
